(19) United States
(12) Patent Application Publication
(10) Pub. No.: US 2014/0364778 A1
(43) Pub. Date: Dec. 11, 2014

(54) FABRIC BASED DEVICE HAVING FEATURES OF VIBRATION, HEATING AND LIGHT

(71) Applicant: Sentrix Technology Limited, Kowloon (HK)

(72) Inventors: Steve Leftly, Christchurch (NZ); Dianne Jones, Christchurch (NZ); Isaac Man, Cheung Sha wan (CN)

(73) Assignee: Sentrix Technology Limited, Kowloon (HK)

(21) Appl. No.: 14/287,081

(22) Filed: May 26, 2014

(30) Foreign Application Priority Data
Jun. 6, 2013 (GB) 1310058.1

(51) Int. Cl. A61H 23/00; A61F 7/00

(52) U.S. Cl. CPC A61H 23/00; A61F 7/00; A61H 2201/0207; A61H 2201/50
USPC 601/18; 601/46

(57) ABSTRACT

A fabric based device (100) having features of vibration, heating and/or light emission. The fabric based device comprises layers of fabric (104) and soft, washable power and signal cables (102), which are encapsulated in fabric layers (104). The power and signal cables (102) provide power to the vibration modules (106) and heating layer of fabric (202). Vibration modules (106) are embedded in encasing material (108) and welded onto the fabric layers (104), thus creating a vibrating and heating fabric based device (100).

### BACKGROUND

1. Field

The disclosed subject matter, in general, relates to the field of vibration and thermoregulation devices for therapeutic use, and more particularly, but not exclusively, to integration of such devices in wearable fabric materials.

2. Discussion of Related Field

It is well known that, imparting of vibration and heat to a part of the body, which is experiencing pain, sprains, stress or strain, helps in relieving such ailments.

Imparting of vibration and heat to the body part is usually carried out using devices, which are configured to generate and impart vibration and heat. When such devices are applied on the ailing body part, for certain duration, the heat and vibration imparted will reduce the ailment by relaxing the muscles of the ailing body part.

When heat is imparted to muscles, it creates higher tissue temperatures, which produces vasodilation. This increases the supply of oxygen, and nutrients, and eliminates carbon dioxide and metabolic waste in the muscles. This situation leads to relief of the ailing body part. Similarly, imparting of vibrations to the muscles, leads to relaxation of muscles and provides relief to the ailing body part.

The benefits of heat energy and vibration for therapeutic uses has lead to creation of several products, which utilizes heat and vibration, either separately or jointly to provide relief to body parts in certain ailing conditions.

Such techniques are disclosed in Mexican patent application MX2007014064, wherein, a therapy patch is disclosed. The therapy patch delivers vibration and heat to the human skin, to provide relief to muscles. Such patches can be attached to the skin, using an adhesive. However usage of such therapy patches proves to be a hassle, as it has to be stuck over the skin and one has to use multiple such patches, is cases of multitude of ailing areas in the body. In addition to the above-mentioned drawback, while using of such patches, the mobility of the user may also be hampered.

In one of the conventional techniques, as disclosed in US patent application US2008027363, there is disclosed a wearable therapeutic device, which comprises of vibrating mechanism to impart vibrations. Such a device is configured such that, it is worn by the user. Such a wearable device is usually configured to be worn only around the waist. Such a device cannot be wrapped around other parts of the body. In addition to this, the configuration of the vibrator and wires to provide power to the vibrator is such that, it forms a bulky device.

Though there are devices, which have incorporated the techniques of providing vibration and heat energy to provide relief to the user, these devices are not able to fully satisfy the need of being used easily and effortlessly by the user. Further, such devices are not known to be waterproof. It cannot be washed using water, without removing the vibrator and the connection wires.

Further, the devices know in the art do not offer flexibility of use and wrapping around the ailing muscle with ease. Furthermore, the vibrators and heat imparting mechanisms are not integrated seamlessly into to the wrap around materials.

In light of the foregoing discussion, there is a need for a technique for integration of vibrators and heat imparting materials seamlessly into wearable fabric.

### BRIEF DESCRIPTION OF DRAWINGS

Embodiments are illustrated by way of example and not limitation in the Figures of the accompanying drawings, in which like references indicate similar elements and in which:

FIG. 1 illustrates the cross sectional view of a fabric based device 100, in accordance with an embodiment;

FIG. 2 illustrates the cross sectional view of an alternate embodiment of a fabric based device 100, in accordance with an embodiment;

FIG. 3 illustrates an alternate cross sectional view of the fabric based device 100, in accordance with an embodiment;

FIG. 4 illustrates a top view of the fabric based device 100, in accordance with an embodiment; and

FIG. S illustrates a device 500, which is used to control certain parameters of the fabric based device 100, in accordance with an embodiment.

### DETAILED DESCRIPTION OF PREFERRED EMBODIMENTS

### I. OVERVIEW

### II. EXEMPLARY SYSTEM

### III. EXAMPLE

### IV. CONCLUSION

### I. Overview

Embodiments relates to the field of vibration, thermoregulation, and light emitting devices for therapeutic use, and more particularly, but not exclusively, to integration of such devices in wearable fabric materials.

In an embodiment, a fabric based device is comprised of vibrator modules and layers of fabric. Cables required to power the vibrator modules and to provide heating to the fabric layer are such that, they can be woven into the fabric. The cables are soft and washable in nature. The cables are encapsulated in the fabric. The vibration module is embedded in the fabric multilayer. The vibration module is housed in a specially designed soft case, which can be made of soft foam or fabric or silicone. A top layer is comprised of a fabric layer. The fabric based device formed of such construction is wearable, having features of vibration and heating. Such fabric based device can be used for therapeutic purposes.

The following detailed description includes references to the accompanying drawings, which form part of the detailed description. The drawings show illustrations in accordance with example embodiments. These example embodiments are described in enough detail to enable those skilled in the art to practice the present subject matter. However, it will be apparent to one of ordinary skill in the art that the present invention may be practiced without these specific details. In other instances, well-known methods, procedures, components, circuits and networks have not been described in detail so as not to unnecessarily obscure aspects of the embodiments. The embodiments can be combined, other embodiments can be utilized or structural and logical changes can be made without departing from the scope of the invention. The following detailed description is, therefore, not to be taken as a limiting sense.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

### II. Exemplary System

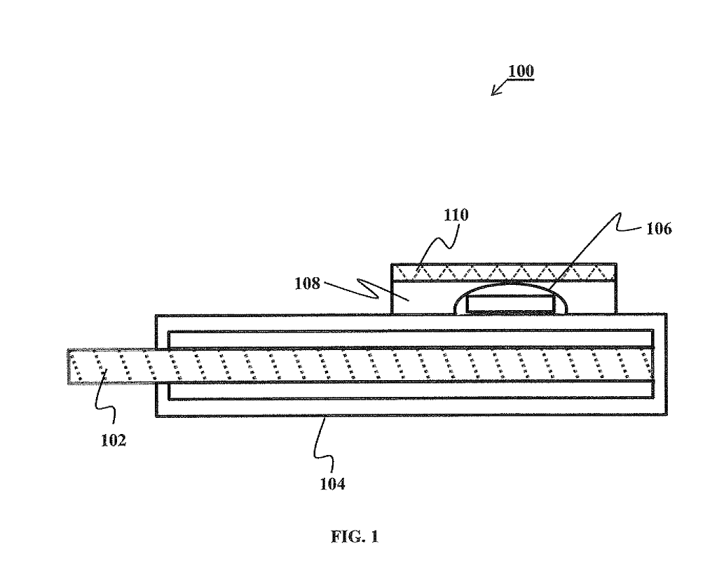

Embodiments disclose a fabric based device comprising a vibration module encased in an encasing material, wherein FIG. 1 illustrates the cross sectional view of a fabric based device 100, in accordance with an embodiment. The fabric based device 100 comprises a vibration module 106. The vibration module 106 is welded onto a fabric layer 104. The vibration module 106 can be encased in an encasing material 108. Although, there is only one vibration module and one fabric layer depicted in the FIG. 1, a person skilled in the art will understand that there can be multiple vibration modules and fabric layers incorporated into the fabric based device.

The encasing material 108 may include, but not limited to, one or more of fabric, foam and silicone. A top layer 110 of the encasing material 108 can be a soft fabric material. One or more supply cables 102 are encapsulated by the fabric layers 104. The supply cables 102 are configured to supply power to the vibration module 106. The supply cables 102 are made of materials, which are washable and soft in texture. This enables the supply cables 102 to be encapsulated within the fabric layer 104.

In an embodiment, the vibration module 106 is embedded into the encasing material 108. The encasing of vibration module 106 in soft silicone material makes the vibration module 106 waterproof and soft. Further, the encased vibration modules 106 is welded onto the fabric layer 104. The welding of vibration module 106 onto the fabric layer 104 can be enabled by adopting polymer bonding techniques.

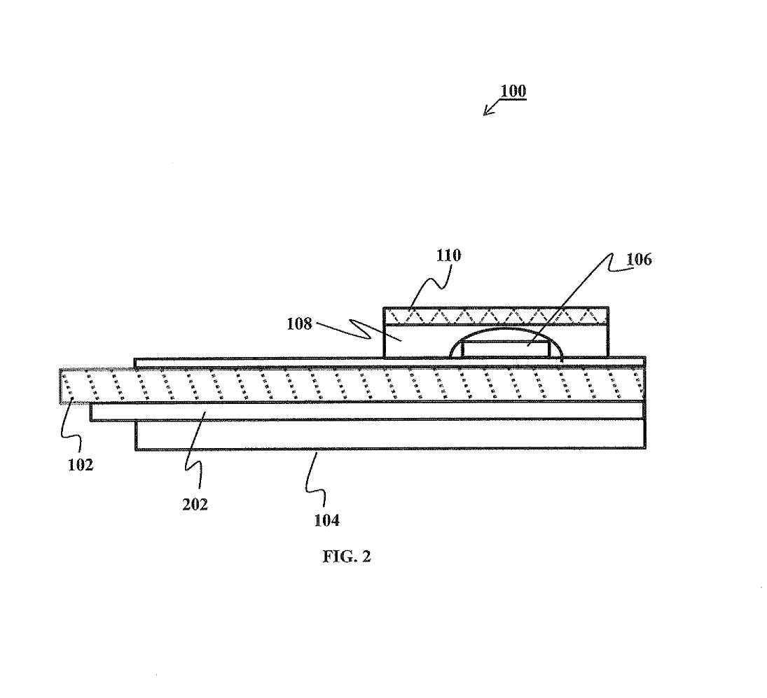

In an embodiment, the fabric based device 100 further comprises a heating layer of fabric. FIG. 2 illustrates the cross sectional view of an alternate embodiment of a fabric based device 100, in accordance with an embodiment. The fabric based device 100 comprises a heating layer 202. The heating layer 202 can be bonded to the fabric layer 104. The heating of the heating layer 202 is enabled through the power supplied by a power source and signal cables 102. In an embodiment, the fabric layer 104 may be used to achieve the functionality of the heating layer and thereby eliminating the need for an additional heating layer 202.

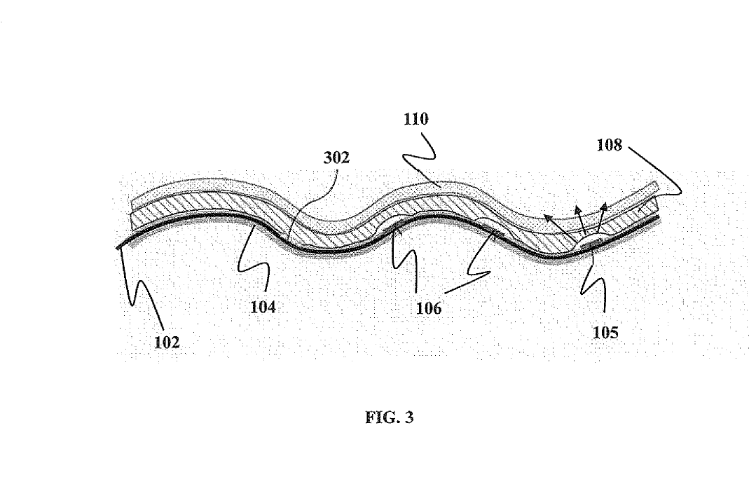

In an embodiment, the fabric based device 100 may be designed to be wearable. FIG. 3 illustrates an alternate cross sectional view of the fabric based device 100, in accordance with an embodiment. Along with vibration module 106, which are embedded in an encasing material 108, a microcontroller 302 is also embedded in the encasing material 108.

The microcontroller 302 is configured to control the operation of the vibration module or the heating layer by executing one or more instructions, which are stored in the microcontroller 302. The instructions stored in the microcontroller 302 may pertain to controlling of the vibration modules 106 and the heating layer 202. The microcontroller 302 is configured to enable switching on and switching off the vibrator modules 106. In an embodiment, the microcontroller 302 is configured to instruct the vibrator module 106 to vibrate at instructed frequencies. Further, the microcontroller 302 is configured to control the amount of power supplied to the heating layer 202. The power supplied to the heating layer 202 is directly proportional to the amount of heat generated in the heating layer 202. The microcontroller 302 is configured to receive instructions from an external device and further, carry out certain predetermined tasks based on the logic embedded in the microcontroller 302.

In an embodiment, batteries can be embedded in the encasing material 108. The encased batteries can be welded onto the fabric layer 104. The batteries can supply power to the vibrator module 106 and the heating layer 202. The supply of power to and from the batteries can be enabled by the power and signal cables 102.

In an embodiment, the power to the vibrator module 106 and the heating layer 202 can be supplied from an external source. The power and signal cables 102 enable the supply of power. The power and signal cables 102 further enable the communication of data to and from the microcontroller 302.

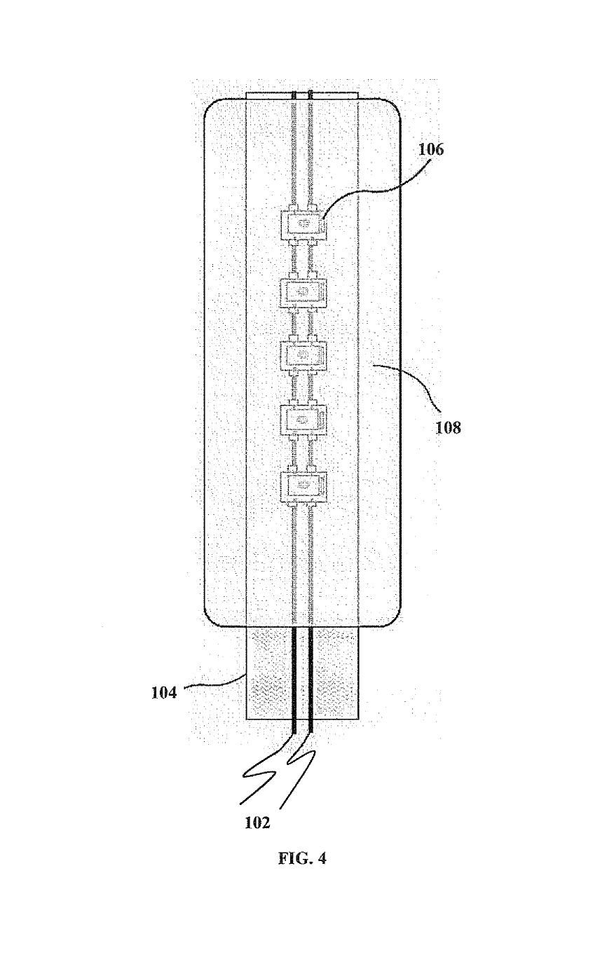

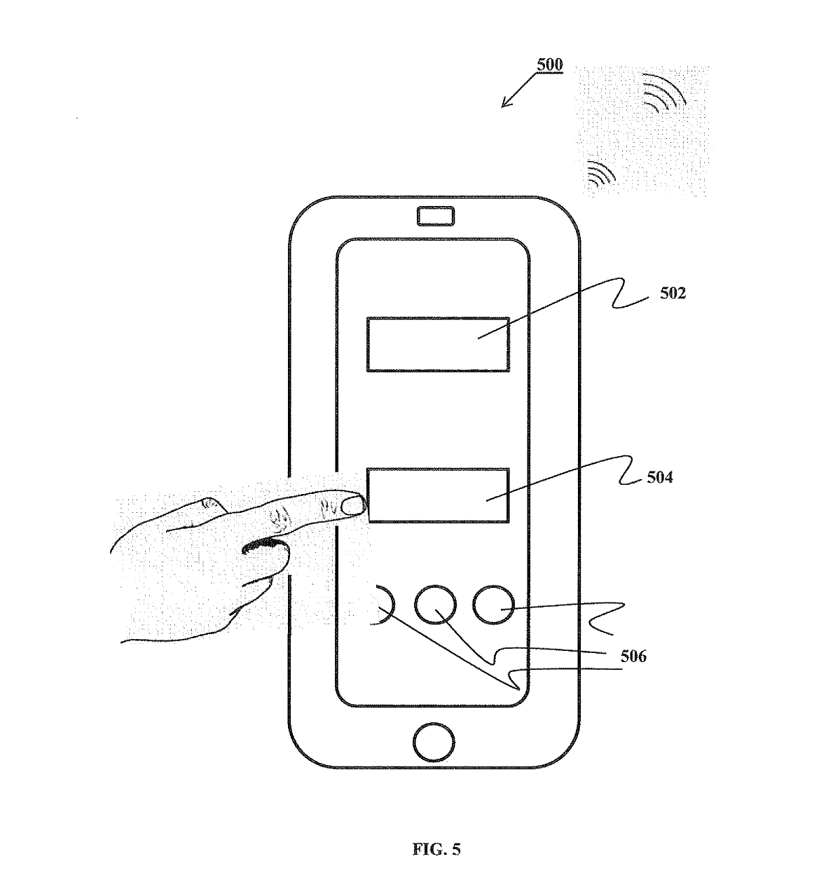

FIG. 4 illustrates the top view of the fabric based device 100, in accordance with an embodiment. The top view of fabric based device 100 is illustrated as a transparent view in FIG. 4. This view shows the fabric layer 104 at the bottom. The power and signal cables 102 are encapsulated in the fabric layers 104. The vibrator modules 106 are embedded in an encasing material 108 and thereupon welded onto the fabric layer 104. The power and signal cables 102 are run through the vibrator modules 106.

FIG. 5 illustrates a device 500, which is used to control certain parameters of the fabric based device 100, in accordance with an embodiment. Device 500 comprises a vibration controller 502, heat controller 504 and preset vibration and heating values 506.

In an embodiment, the device 500 can include a touch interface device. The sliding of a finger on the vibration controller 502 in a predetermined direction can increase or decrease the level and degree of vibration imparted by the vibration module 106. The sliding of a finger on the heat controller 504, in a predetermined direction, can increase or decrease the heat in the heating layer 202. Choosing the preset vibration and heat values 506 sets the vibration and heat values as per the preset values.

In an embodiment, the device 500 is capable of communicating with the microcontroller 302. The microcontroller 302, upon receiving a request, executes the instructions, based on the preset logic in the microcontroller 302.

In an embodiment, the device 500 may communicate with the microcontroller 302 through a wired medium, wireless medium and a combination thereof.

In an embodiment, the fabric based device 100 can include one or more Light Emitting Diodes (LED) 105 welded to the fabric layer 104. Further, the LED's 105 may be encapsulated using the encasing material 108.

In the instant embodiment, the encapsulated LED's 105 may be configured to emit blue light to enhance the healing process or provide other therapeutic effect like pain relief or relaxation.

### III. Example

The bottom layer of the fabric based device 100 is a fabric layer 104. The power and signal cables 102 are encapsulated in the fabric layers 104. The vibrator module 106 are embedded in an encasing material 108. The encasing material can be made of silicone and foam or any other suitable materials. The encasing material 108, makes the vibrator module 106 waterproof and soft. Microcontroller 302 and batteries can also be embedded in a similar technique. A heating layer 202 is also provided as a fabric layer. The heating layer 202 can be a fabric material. The encasing material with the embedded vibrator module 106, microcontroller 302 and batteries is welded onto the fabric layer 104. This configuration forms the fabric based device 100.

The fabric based device 100, can be wound on a body part, similar to a crepe bandage and thereupon, device 500 can be used to control the fabric based device 100 to carry out the desired functions. The functions can pertain to vibration, heating and other preset configurations.

The power supplied to the fabric based device 100 can be from the batteries embedded in the fabric based device 100 and/or from an external source.

### IV. Conclusion

In light of the above disclosure, it is evident that, the present invention has many advantages over existing technologies. Some of those advantages are mentioned below:

The configuration of the fabric is such that, a vibrating, heating and light emitting fabric based device is created.

The fabric based device can be used in soft garment and apparel applications.
The configuration of the fabric based device enables it to be washable and durable.
The power and signal cables used in the fabric based device are soft and washable,
Unlike products found in prior art, the fabric based device is easily wearable in any desired part of the body and there are no hard parts in the fabric.
The fabric based device can be used for a wide range of applications such as performance, medical rehabilitation, muscle conditioning, pain relief, relaxation and other applications.

The processes described above is described as sequence of steps, this was done solely for the sake of illustration. Accordingly, it is contemplated that some steps may be added, some steps may be omitted, the order of the steps may be re-arranged, or some steps may be performed simultaneously.

The example embodiments described herein may be implemented in an operating environment comprising software installed on a computer, in hardware, or in a combination of software and hardware.

Although embodiments have been described with reference to specific example embodiments, it will be evident that various modifications and changes may be made to these embodiments without departing from the broader spirit and scope of the system described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention. Thus the scope of the invention should be determined by the appended claims and their legal equivalents rather than by the examples given.

We claim:

1. A fabric based device comprising:
a fabric layer;
a soft washable power and signal cables encapsulated in the fabric layer; and
at least one vibration module embedded ii an encasing material and welded onto the fabric layers.

2. The fabric based device according to claim 1, further comprising a heating layer configured to receive energy and produce heat.

3. The fabric based device according to claim 2, wherein the soft and washable power and signal cables are configured to supply power to the vibration modules and the heating layers.

4. The fabric based device according to claim 1, wherein the encasing material is chosen from a group consisting of foam, fabric and silicone.

5. The fabric based device according to claim 1, wherein encasing of the at least one vibration modules makes the vibration modules waterproof.

6. The fabric based device according to claim 1, further comprising a microcontroller, wherein, the microcontroller is encased in the encasing material and welded onto the fabric layers.

7. The fabric based device according to claim 6, wherein the microcontroller is configured to receive one or more requests and execute instructions based on programmed logic of the microcontroller.

8. The fabric based device according to claim 6, wherein the microcontroller is configured to receive the instructions wirelessly.

9. The fabric based device according to claim 6, wherein the microcontroller is configured to control the at least one vibration module and heating layer.

10. The fabric based device according to claim 6, further comprising an external device, wherein the external device is configured to communicate with the microcontroller to control the at least one vibration module and heating layer.

11. The fabric based device according to claim 1, further comprising one or more Light Emitting Diodes in the encasing material and welded onto the fabric layers.

12. The fabric based device according to claim II, wherein the Light Emitting Diodes are configured to emit blue light.

13. The fabric based device according to claim 1, wherein the fabric based device is wearable and washable.
